(19) United States
(12) Patent Application Publication
(10) Pub. No.: US 2012/0158123 A1
(43) Pub. Date: Jun. 21, 2012

(54) POLYMER STENT

(75) Inventor: Alexander Borck, Aurachtal (DE)

(73) Assignee: BIOTRONIK AG, Buelach (CH)

(21) Appl. No.: 13/309,276

(22) Filed: Dec. 1, 2011

Related U.S. Application Data
(60) Provisional application No. 61/423,124, filed on Dec. 15, 2010.

(51) Int. Cl. A61F 2/82; B29C 35/08

(52) U.S. Cl. 623/1.15; 264/400

(57) ABSTRACT

The invention relates to a stent, comprising or consisting of amorphous polylactide. The invention relates further to the use of amorphous polylactide for stabilizing a stent and a method for producing a corresponding stent.

### CROSS REFERENCE TO RELATED APPLICATIONS

This invention claims benefit of priority to U.S. patent application Ser. No. 61/423,124, filed Dec. 15, 2010; the contents of which are herein incorporated by reference in their entirety.

### TECHNICAL FIELD

The invention relates to a stent (a vessel support) comprising or consisting of amorphous polylactide. The invention relates further to the use of amorphous polylactide for stabilizing a stent and a method for producing a corresponding stent.

### BACKGROUND

Polymer-based stents have a lower strength with respect to metallic stents. In case of a base body composed of polymers, the minimally necessary radial force has to be compensated by more material and thus greater wall thicknesses. Thicker wall thicknesses and struts verifiably result in worse clinical results, e.g., due to increased neointima proliferation.

From the prior art, the so-called AMS stent is known (EP 1 270 023; EP 1 632 256; EP 1 419 793). This stent has deformation properties which lie between the ones of polymers and the ones of stainless steel alloys. At the same time, the stent is degradable in the body (bioresorbable) which is desired for many uses.

Further, biologically degradable stents on the basis of polymers are known. In the prior art, for reasons of biocompatibility, stents made of polylactide (PLA) are used or stents which contain an adequate proportion of polylactide. With respect to their mechanical behavior, polymers such as polylactide can be divided in brittle, plastically deformable and elastically deformable. The deformability of the material is defined by the position of the two following limits: The yield strength and the fracture strength.

The yield strength is a material parameter which defines the stress up to which a material does not show any visible plastic deformation during a uniaxial and moment-free tensile load. When the yield strength is exceeded, the material will not return to its original shape, but an elongation remains. Up to this yield strength, an elastic, reversible deformation occurs.

At the fracture strength, the deformation stops and the material fractures.

A stent should principally be able to be fitted onto a dilatation device, i.e., the stent must be crimpable and, at the same time, dilatable in order to be able to allow a circumferential expansion after the insertion at its destination site and thus to provide for an adequate is lumen enlargement. Crimpability and dilatability require a sufficient plasticity.

On the other hand, a stent must also be able to exert a sufficient supporting force without elastic or plastic recovery (low recoil). These requirements are in contradiction to each other. The deformation properties of materials are functions of time, shearing forces and temperature. Low elastic or plastic deformation occurs only in case of brittle materials which therefore cannot be crimped or dilated in a sufficient manner. This disadvantage is compensated in the prior art in some cases by very low deformation speeds (during dilatation).

This results in that known polymer stents, in particular such made of PLA, should be dilated only with reduced speed. Usually, 2 bar within 5 seconds is specified as upper limit for the pressure increase during the dilatation, which is extremely unpleasant for patients.

Principally, when introducing an implant, a rapid intervention is desirable because thereby, the burden on the cardiovascular system can be kept low due to a very short interruption of the blood supply and, overall, a short intervention time burdens the organism less than a longer one.

### SUMMARY

Against this background, it was the object of the invention to provide a stent which has good mechanical properties, wherein the wall thicknesses (strut thicknesses) should lie within a medically acceptable range. At the same time, the stent has to be easy to handle during the implantation and, in particular, the dilatation should take place rapidly and the stent should be crimpable in a sufficient manner.

As mechanical property, the stent as implant should have in particular a sufficient supporting force with respect to strength and duration of the supporting effect on the vessel. Further, in the meaning of the object it was preferred that the stent is bioresorbable and it was particularly preferred in the meaning of the object that the degradation of the stent in the course of the bioresorption begins primarily in regions which are not essential for the radial strength of the stent.

The above described object of the invention is solved by a stent comprising, consisting essentially of or consisting of polylactide, wherein the proportion of amorphous polylactide ≧80% by weight, based on the total mass of the polylactide contained in the stent. The material is in a metastable state, wherein the material has the ability and the ambition to recrystallize.

Preferably, a stent according to the invention comprises a proportion of ≧90% by weight of amorphous polylactide based on the total mass of the polylactides contained in the stent.

Surprisingly, it was found that stents with a high content of amorphous polylactide fulfill the requirement combination of crimpability and dilatability (sufficient plasticity) and supporting force for the vessel formulated in the context of the object in a particular manner, namely in that amorphous PLA, in the course of the dilatation, (at least partially) changes into crystalline form.

P(L)LA involves in most applications a semi-crystalline polymer. This means that normally 70% of the polymer is organized in crystalline domains, while approximately 30% are present in amorphous form. In usual processing forms from a solvent, the PLA material has the possibility to align itself structurally over the duration of the evaporation in such a manner that a very high proportion is present in crystalline form. While amorphous PLA is metastable under normal conditions, the crystalline form is the thermodynamically more stable state. Therefore, to avoid uncontrolled conversion processes, materials from PLA are often tempered to increase the proportion of thermodynamically more stable, crystalline PLA.

However, it is technically also possible, e.g., when processing from the melt, to significantly increase the proportion of amorphous PLA: This can take place, for example, by cooling the polymer melt very rapidly (e.g. by quenching) so that the major part (preferred the entire material) is present in amorphous form (undercooled melt). At room temperature, this state can be conserved for at least 12 month, further preferred 18 month and particularly preferred 24 months. It is necessary here that the material is not excessively heated. The storage preferably takes place at room temperature, wherein temperature increases to 40° C. are allowed only for a few hours (<2 h). Here, the humidity should not exceed a relative humidity of 40-45%. The preservation capability of this metastable state depends, among other things, on the polymer chain length. Preferred polymer chain lengths in this connection are average molecular weights of 150-700 kDA, wherein average molar masses of 200-500 kDa are preferred. Only chain lengths of this size are metastable even up to 40° C. and over extended periods (up to 1 year). Due to their greater mobility, short chains are prone to reorganization or, respectively, recrystallization.

In case of a corresponding energy supply, the amorphous PLA can change into the crystalline state due to the increase of the polymer chain mobility.

This reorganization process is exothermic. This is also called recrystallization. The energy supply can take place thermally (tempering) or also mechanically.

In the context of the present invention, the term “polylactide” (PLA) relates to lactide chains, wherein all the monomer units have the L-form or the D-form of the lactide or can be present in any mixture of the L- and D-form up to and including the racemate. Preferred in the meaning of the invention is PLA as the L-form (PLLA). The advantage of PLLA is in particular an excellent bioresorbability and a good commercial availability. The use of only one stereoisomer ensures that the material has the ability of recrystallization.

In its initial state, the stent according to the invention is flexible and easily plastically deformable due to the high content of amorphous PLA. After the dilatation, the stent is subjected to a structural change which occurs primarily at the regions which are mechanically loaded during the dilatation. Due to the energy introduced into the stent during the dilatation, in particular in these regions, a considerable portion of the amorphous PLA changes into the crystalline state. The crystalline state means that said PLA regions embrittle with respect to the amorphous state so that material becomes locally less flexible and thereby can exert an improved supporting effect on the vessel. This means that the stent according to the invention is easily deformable prior to the mechanical loading during the dilatation; after the dilatation, due to the transition of portions of the PLA into the crystalline state, the stent loses this ability (at least partially); however, the stent provides instead an improved supporting effect.

A further advantage of the configuration of the stent according to the invention is that a “re-embrittling” can take place: Regions of the stent which are not subjected to a high mechanical load can still comprise significant proportions of amorphous PLA. If said regions are subjected to a significant mechanical load during the intended use, the energy introduced thereby can initiate a transition of further proportions of the PLA into the crystalline state, which improves the supporting effect at the points which are particularly mechanically loaded.

It is preferred that the stent according to the invention is bioresorbable. In this context, bioresorbable means that within a time period of 1-3 years and under normal conditions of use, the stent can be completely degraded by the (human) body.

Bioresorbability can be achieved by the use of suitable materials, e.g., by a combination of lactide, in particular L-lactide, and bioresorbable magnesium alloys such as, e.g., alloys as they are described in EP 1 632 256 of Gerold et al. and EP 1 419 793 of Gerold et al. However, it is preferred that the stent consists to a large extent—particularly preferred s completely—of polylactide, in particular poly-L-lactide. Also, further components of a preferred stent can be (bioresorbable) coatings which further improve the body compatibility, and/or active ingredient coatings/inclusions such as, e.g., biolimus, sirolimus or paclitaxel.

In case of the preferred bioresorbable stents according to the invention, in particular such stents which (substantially) consist of PLLA, it is an advantage that during the dilatation, the sections of the stents which are particularly mechanically loaded change in higher proportions into the crystalline form of PLLA as this is the case for the ones which are subjected to a lower mechanical load: Even under normal conditions within the body such as, e.g., at 37° C., regions which have not been mechanically deformed remain amorphous over, e.g., 1-2 months. Said amorphous regions show higher water absorption than crystalline regions. Water facilitates the degradation of the stent in the course of the biodegradation. Accordingly, the resorption of the stents according to the invention begins at regions which are of less importance for maintaining the supporting force. At the same time, the basic scaffold becomes more flexible. Water can more easily penetrate into amorphous regions. Also, the latter have a lower density compared to crystalline domains. The hydrolytic degradation starts due to the higher water absorption or, respectively, the stronger swelling behavior. Due to the chain breakages and shortening of the polymer chains, the material becomes more flexible.

Preferred is a stent according to the invention which is sterilized. In this context, sterile means a method that is suitable according to the medical equipment law and is acceptable for the use of the implant in or on the human body.

It has to be considered here that the sterilization has to be carried out without an excessive proportion of the amorphous polylactide transitioning into the crystalline form. Accordingly, sterilization with ethylene oxide (ETO) is usually not recommended.

Preferred sterilization methods for a stent according to the invention are: e-beam or gamma radiation, wherein the implant should be cooled on ice (0° C.) during the sterilization process.

Of course, it is appropriate to use only sterile stents as implants. As indicated, the sterilization is to be performed in such a manner that the particular properties, in particular the possibility of the transition from the amorphous form into the crystalline form, are maintained for the polymer.

Preferred is a stent according to the invention for which the polylactide is selected such that during storage at 25° C. for 12 months, ≦10% by weight of the amorphous polylactide convert into crystalline polylactide.

Particularly preferred in the context with the storage-stable stent according to the invention is that the polylactide has an average molecular weight of 150-700, preferably 200-500 and in particular of 250-350 kDa. These values relate to the finished stent or, respectively, the stent after the sterilization.

Preferred is a stent according to the invention, wherein polylactide can be manufactured by extrusion of polylactide having an inherent viscosity (iv-value) of ≧3.3 dl/g and preferred ≦4.3 dl/g. The iv-values are determined at 25° C. in chloroform and a concentration of 0.1%. The iv-values are present after the extrusion.

Polylactide in this configuration, in particular PLLA, is particularly easy to handle and provides the stents made therefrom with particularly good mechanical properties.

Further preferred is a stent according to the invention, wherein 1-10% by weight, preferably 2-6% by weight of the polylactide based on the total mass of the polylactide has a molar mass of 400 Da-3000 Da after the extrusion.

This “size blend” of polylactide with small lactide polymers/oligomers has proved to be particularly readily bioresorbable. In addition, the shorter polymer chains act as polymeric softeners and thus increase additionally the flexibility of the polymer scaffold. The higher flexibility of the shorter chains has also a great influence on the recrystallization. By the blend of the longer chains with shorter chains, the amount of energy necessary to start the recrystallization is significantly reduced.

Also preferred according to the invention is a stent which is crimped (fitted) onto a balloon catheter. Said stent preferred according to the invention (in combination with the balloon catheter) is ready for implantation.

It is preferred here that the stent is fitted very slowly at 5-15° C., particularly preferred at 10° C., onto the balloon catheter. This process takes preferably 0.5-3 minutes, particularly preferred 1-2 minutes.

The fitting has to be carried out very carefully because during this process step there is the is risk that the polymer material prematurely crystallizes (at least partially) due to the deformation.

Of course, it makes sense for the stents according to the invention to store them under suitable conditions. Excessive humidity at temperatures above 40° C. facilitates the transition of amorphous PLA (in particular PLLA) into crystalline PLA. In this case, the stent would become brittle and could not be dilated with a sufficient speed.

Consequently, a stent according to the invention is preferred which, when used as implant in a person or an animal, can be dilated after insertion into the body at a pressure increase of ≧5 bar per 5 seconds, preferred ≧7 bar per 5 seconds and particularly preferred ≧10 bar per 5 seconds.

This property can be achieved according to the invention by a sufficiently high proportion of P(L)LA in the amorphous state, wherein the stent preferably consists—as described above—(substantially) of PLLA.

Of course, the stents according to the invention are suitable for being used in the field of medicine. This applies in particular to the preferred variants of the stents and the use as implant.

Also part of the invention is the use of amorphous polylactide for stabilizing a stent by transitioning from the amorphous form into the crystalline form during the dilatation of the stent.

By said usage, the particular properties of the different states of the polylactide are utilized in an advantageous manner for the use of a stent. In this manner, the above described advantages with respect to the mechanical properties of the stent can be achieved.

The invention relates further to a method for producing a stent according to the invention, comprising the steps:

a) providing polylactide, wherein the polylactide is present in amorphous form in a proportion of at least >80% by weight based on the total mass of the polylactide, and

b) forming a stent made of the polylactide or by using the polylactide under such conditions that the proportion of amorphous polylactide is ≧80% by weight based on the total mass of the polylactide contained in the stent.

With said method according to the invention, the stents according to the invention can be produced. It is of course preferred within the context of this method that a further step, namely the sterilization, is carried out. In this case, as described above, the sterilization is to be carried out such that a sufficiently high proportion of amorphous polylactide is maintained.

Further, in step b), care must be taken to ensure that during forming the stent no excessive energy is introduced into the stent, regardless whether the energy is of mechanical or thermal nature.

Accordingly, it is recommended to carry out a laser cutting process for forming the stent (in its non-dilated form) by means of a UV laser, wherein the UV laser is preferably a femtolaser.

Modern UV lasers are able to provide laser energies of up to one J/pulse, wherein some lasers achieve pulse rates of up to 300 Hz. Devices with pulse rates of 50 Hz are preferably suited for the method according to the invention.

For purposes of the invention, the proportion of amorphous polymer or crystalline polymer o can be determined by means of dynamic differential scanning calorimetry (DSC).

Through the phase transition from amorphous to crystalline associated with a heat tone, the polymer can be readily characterized. By means of a heating-up ramp, the proportion of residual crystallinity can be determined when the material changes into its thermodynamically stable state. When the material melts in the DSC and is cooled extremely rapidly (>50 K/min.), the maximum proportion of crystallization heat can be determined which is inherent in the material (thus in the maximally amorphous state).

Viscosity measurements for the above mentioned viscosity values are carried out under the following conditions: Measurement in chloroform at 25° C. with a polymer concentration of 0.1%.

### DESCRIPTION OF THE DRAWINGS

The invention is further explained hereinafter by means of the figures and by means of an example:

FIG. 1 illustrates a heat flow curve as a function of the temperature by means of dynamic differential scanning calorimetry (DSC) of an amorphous polymer sample which was rapidly cooled after the extrusion (cooling rate 50° C./min.)

FIG. 2 illustrates a corresponding diagram of the same material which was previously mechanically deformed. The deformation of the material was 300-400% longitudinal elongation.

### DETAILED DESCRIPTION

By comparing the two diagrams to each other, the influence of the mechanical load on amorphous material is made clearly visible by means of DSC measurements.

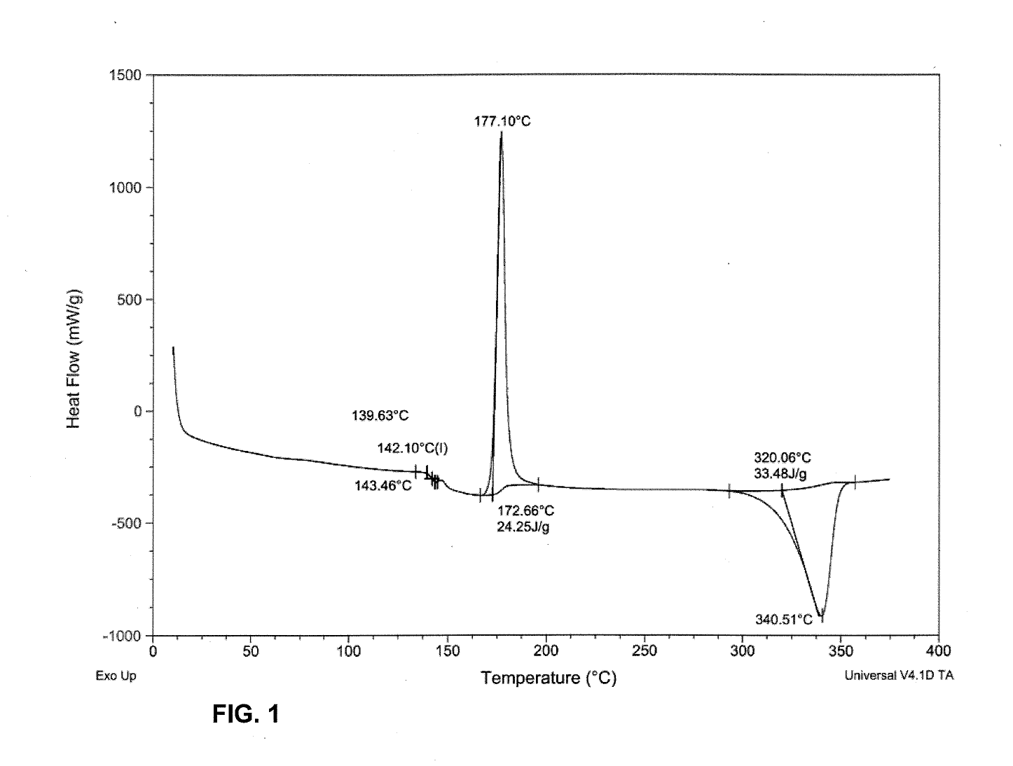

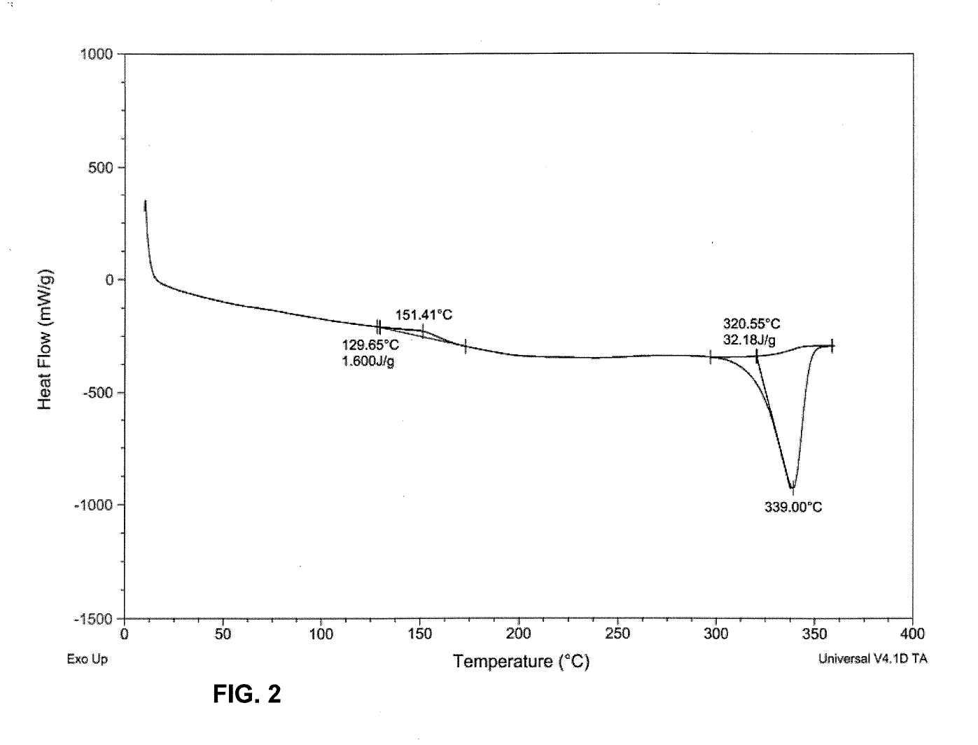

Amorphous polymers such as PLLA show an exothermic recrystallization peak in the thermogram. In contrast, if the material is tempered or treated in such a manner that a thermostable state is achieved, no exothermic peak appears in the first run (1^{st} heating phase) and thus no recrystallization. This is clearly shown in the two figures of the example: FIG. 1 shows the glass transition of the polymer at 142° C., the recrystallization of the originally amorphous polymer in the form of a sharp peak at 177° C., and the melting process at 320° C. In contrast to FIG. 1, FIG. 2 shows a curve progression for a material which was previously deformed by 300-400%. Here, at 151° C., only a (residual) recrystallization is shown and the melting process is shown at 320° C.

At 151° C., the recrystallization is almost completed; only a residual heat tone of 1.6 J/g can be seen. In contrast to that, the melting behavior is very similar in both curves (melting process at 320° C.).

Regions of the material, for which the temperature-dependent heat flow behavior is illustrated in FIG. 2, and which have not been subjected to mechanical load, showed a thermal behavior corresponding to FIG. 1. Transition regions with low mechanical load showed values also with regard to the recrystallization which values, with respect to the heat tone at 150-170° C., lay between the values from FIGS. 1 and 2.

### EXAMPLE

From PLLA (batch L210, Boehringer Ingelheim) with an inherent viscosity of 3.3 dl/g-4.3 dl/g (measured for a concentration of 0.1% in chloroform at 25° C.), a tube was extruded by means of an extrusion method. The tube has an inner diameter of 0.6-1 mm, preferably 0.8 mm. The wall thickness is 70-250 μm, preferably 100-200 μm, further preferred 150 μm. Extrusion methods are well known to the person skilled in the art.

The extruded PLLA material comprised 5% by weight of LLA oligomers/polymers with an average molar mass of 1,500 g/mol.

It was ensured that during the extrusion process and afterwards, the tube was not heated above room temperature. Subsequently, a stent was formed by means of a laser cutting process with a femtolaser. During laser cutting, the heat influence in the region of the cuts took place only on a small zone of the respective material.

After this, the stent was crimped at 10° C. onto a balloon catheter and sterilized by means of e-beam. The instrument was placed on ice during the sterilization. The crimping process took 2 minutes. It was ensured that the mechanical load on the stent was low so that no premature crystallization of the amorphous material took place.

After it was fitted onto the balloon catheter, 90% of the PLLA material was present in amorphous form. With the stent of the example, a dilatation at a pressure increase of 10 bar within 10 sec can be carried out in a human body without generating disadvantageous results.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

What is claimed is:

1. A stent, comprising polylactide, wherein the proportion of amorphous polylactide is ≧80% by weight, based on the total mass of the polylactide contained in the stent.

2. The stent according to claim 1, wherein the proportion of amorphous polylactide is ≧90% by weight, based on the total mass of the polylactide contained in the stent.

3. The stent according to claim 1, wherein the polylactide has an average molecular weight of 150-700, preferably 200-500 and in particular 250-350 kDa.

4. The stent according to claim 1, wherein the polylactide is L-polylactide.

5. The stent according to claim 1, wherein the stent is bioresorbable.

6. The stent according to claim 1, wherein the stent is sterile.

7. The stent according to claim 1, wherein the polylactide is selected such that during storage at 25° C. for 12 months, ≦10% by weight of the amorphous polylactide converts into crystalline polylactide.

8. The stent according to claim 1, wherein the polylactide can be produced by extrusion of polylactide with an inherent viscosity of ≧3.3 dl/g and preferred ≦4.3 dl/g after the extrusion.

9. The stent according to claim 1, wherein 1-10% by weight, preferably 2-6% by weight of the polylactide based on the total mass of the polylactide has a molar mass of 400 g/mol-3,000 g/mol after the extrusion.

10. The stent according to claim 1, wherein the stent is crimped onto a balloon catheter.

11. The stent according to claim 1, wherein the dilatation of the stent after insertion into the body can take place at a pressure increase ≧5 bar per 5 seconds.

12. The stent according to claim 1 for use in the field of medicine.

13. The stent according to claim 1, wherein the stent consists essentially of polylactide.

14. The stent according to claim 13, wherein the stent consists of polylactide.

15. A use of amorphous polylactide for stabilizing a stent by transitioning from the amorphous form into the crystalline form during the dilatation of the stent.

16. A method for producing a stent according to claim 1, comprising the steps:
a) providing polylactide, wherein the polylactide is present in amorphous form in a proportion of at least ≧80% by weight based on the total mass of the polylactide, and
b) forming a stent from the polylactide or by using the polylactide under such conditions that the proportion of amorphous polylactide is ≧80% by weight based on the total mass of the polylactide contained in the stent.

17. The method according to claim 16, wherein the step b) takes place by using a femtolaser.
